(19) United States
(12) Patent Application Publication
(10) Pub. No.: US 2005/0250156 A1
(43) Pub. Date: Nov. 10, 2005

(54) Detection of acute myocardial infarction biomarkers

(76) Inventors: Ronald John Shebuski, Bergland, MI (US); Arthur Raymond Kydd, St. Paul, MN (US); Hiroshi Nomura, Shorewood, MN (US)

Correspondence Address: ALTERA LAW GROUP, LLC, 6500 CITY WEST PARKWAY, SUITE 100, MINNEAPOLIS, MN, 55344-7704, US

(21) Appl. No.: 10/979,771

(22) Filed: Nov. 1, 2004

Related U.S. Application Data
(60) Provisional application No. 60/516,655, filed on Oct. 31, 2003.
(60) Provisional application No. 60/516,656, filed on Oct. 31, 2003.
(60) Provisional application No. 60/516,654, filed on Oct. 31, 2003.

(51) Int. Cl. G01N 33/53; G01N 33/573; C12M 1/34

(52) U.S. Cl. 435/7.1; 435/287.2

(57) ABSTRACT

The present invention relates to medical devices and methods for early detection of acute myocardial infarction in a patient. In particular, the invention relates to a device and method for detecting the presence of biomarker analytes in a specimen which are indicative of a potential acute myocardial infarction in the patient.

### CROSS-REFERENCE TO RELATED APPLICATIONS

This application claims the benefit of U.S. Provisional Patent Application Ser. No. 60/516,655 filed Oct. 31, 2003 (Shebuski et al., “Detection of Acute Myocardial Infarction Precursors”), Ser. No. 60/516,656 filed Oct. 31, 2003 (Nomura, “Method and Apparatus for Body Fluid Analysis Using Surface-Textured Optical Materials”), and U.S. Provisional Patent Application Ser. No. 60/516,654 filed Oct. 31, 2003 (Nomura, “Plasma Polymerization of Atomically Modified Surfaces”), which hereby are incorporated herein by reference thereto in their entirety.

### FIELD OF THE INVENTION

The present invention relates to medical devices and diagnostic methods for the early detection of acute myocardial infarction in a patient. In particular, the invention relates to a device and method for timely and sensitive detection of specific analytes in a fluid sample, such as a blood specimen which may be indicative of a potential or impending acute myocardial infarction in the patient.

### BACKGROUND OF THE INVENTION

Cardiovascular disease (CVD), despite dramatic improvements in diagnosis and therapy, is still the leading cause of death in the world with more than $55 billion spent on cardiac care therapeutics each year. It is estimated that this will increase at an approximate ten percent (10%) annual rate for the next ten (10) to fifteen (15) years. In addition, U.S. government statistics indicate that $5 billion or more of unnecessary medical costs are spent each year on the assessment of non-cardiac (i.e., false positive) cases in hospital emergency departments. As the population ages, this expenditure is expected to grow.

Because of these large and unnecessary utilization costs, there is a need to provide an early assessment of disease risk to allow for rule-out of acute myocardial infarction (AMI) and prevention and early intervention with therapeutics in patients with authentic AMI. The laboratory and imaging diagnostics industries will have a substantial impact on the early detection and selection of heart disease candidates for management.

The “rapid-test” cardiac assay market worldwide is expected to achieve an average annual growth rate of twenty percent (20%) to twenty five percent (25%) for a number of years, driven by a host of newly identified biomarkers indicative of impending AMI, such as high sensitive C-reactive protein (hsCRP), heart type fatty acid binding protein (H-FABP), myeloperoxidase (MPO), brain natriuretic peptide (BNP), P-selectin (soluble and membrane bound), soluble CD40 ligand (sCD40L), glycoprotein IIb/IIIa (GPIIb/IIIa), prothrombin fragment 1.2 (PTF1.2), D-dimer (DD), thrombin-antithrombin II (TAT), beta-thromboglobulin (BTG), platelet factor 4 (PF4), platelet/endothelial cell adhesion molecule 1 (PECAM-1), soluble fibrin, glycogen phosphorylase-BB, thrombus precursor protein (TPP), Interleukin-1 receptor family/ST2, Interleukin 6 (IL-6), Interleukin 18 (IL-18), placental growth factor (PIGF), pregnancy-associated plasma protein A (PAPP-A), glutathione peroxidase, plasma thioredoxin, Cystatin C, serum deoxyribonuclease I, and ATP/ADP.

Additionally, standard cardiac biomarkers indicative of established AMI such as troponin I and T (TnI/TnT), creatinine kinase-MB isoform (CK-MB) and myoglobin (MYO) have been developed as rapid bedside point-of-care (POC) tests. The possible involvement of infectious disease agents in heart disease offers another opportunity for in vitro diagnostics. For the immediate future, the search is ongoing for blood tests that can provide immediate results that are specific, sensitive and accurate to solve the problems of early CVD diagnosis.

The rule-out of acute coronary syndrome (ACS) in chest pain presentations in the emergency room is important. Each year, approximately eight million people in the United States present to the emergency room with chest pain. Approximately 2.5 million are suffering from some form of acute coronary disease and 5.5 million are experiencing non-threatening symptoms such as heartburn, indigestion, stomach cramps or gastro-esophageal reflux disease (GERD). Approximately 2.5 million low-risk chest pain patients are unnecessarily admitted to the hospital as a precaution and at a significant cost to the U.S. healthcare system. In addition, only fifty percent of the patients experiencing ACS receive appropriate therapy in a timely fashion, and this delayed therapy can result in permanent heart muscle damage and greater total cost for patient care.

Chest pain patients include those with deep vein thrombosis (DVT) where the pain has migrated to the chest, back or stomach, and symptoms may be similar to that of ACS. Each year, 600,000 patients will experience venous thromboembolism with perhaps as many as 200,000 dying from blood clots that obstruct blood flow to the lungs (pulmonary embolism). Although the diagnoses of ACS, such as AMI, will receive major attention, DVT and pulmonary embolism are serious conditions that could be avoided with early diagnosis and treatment.

Acute coronary syndromes, such as unstable angina and non Q-wave AMI, are well known to involve the participation and interaction of blood platelets and pro-coagulant proteins to form a thrombus or blood clot. Thrombi/blood clots are often precipitated by acute rupture of an underlying atherosclerotic plaque in which the thin fibrous cap of the atherosclerotic lesion ruptures, exposing surfaces and cells that promote platelet and coagulation activation in an attempt to repair the damage. Acute plaque rupture is highly recognized as the primary cause of acute thrombus formation and complete occlusion of the vessel may result in irreversible ischemic damage to the cardiac tissue supplied downstream from the obstruction. Often the patient does not exhibit complete occlusion of the vessel but has a substantial lesion (greater than 90%) and thus is at extremely high risk of complete blockage and eventual acute myocardial infarction.

Total or sub-total occlusion of a major coronary blood vessel results in sub-sternal chest pain with classic transmittance of the pain to the left extremities. Often the pain is cyclical in nature and is most likely the result of acute thrombus formation occurring at the ruptured lesion site that periodically resolves with restoration of blood flow to the downstream cardiac tissue. This pattern, known as unstable angina, may repeat itself for hours on end and often these patients are admitted into the cardiac chest pain unit and eventually taken to the diagnostic cardiac catheterization laboratory to determine location and severity of a possible coronary lesion.

Non-cardiac chest pain is primarily due to GERD that affects a large portion of the population. However, serious cardiac events must be “ruled-out,” such that patients at risk of an eventual AMI are not released. Thus, there is a need for new diagnostic tests that provide earlier assessment of the factors contributing to an eventual AMI.

It is difficult to separate cardiac from non-cardiac events for the millions of people presenting with chest pain. The symptoms are identical. Chest pain occurs periodically, electrocardiogram (EKG) profiles may not be remarkable, and in early diagnoses (early in the ischemic condition), cardiac protein levels (e.g., TnI, CKMB and MYO) are not yet elevated. These “late” markers, extensively used today, indicate cardiac tissue necrosis, damage that could have been prevented with earlier indications of thrombus and earlier treatment. At present, the emergency room triage process involves serial evaluation of EKG patterns along with these late cardiac markers, over a twenty four hour observation period, as the patient is moved from the emergency room to the hospital's chest pain unit.

The earlier that an intervention, either pharmacological or mechanical, can be started to interrupt or halt the process of myocardial cell death (infarction), the greater the benefit to be realized by the patient. More rapid and specific diagnostic tests that determine the presence of critical cellular and soluble proteins involved in the disease process are required.

Currently, no tests are available in a readily usable format that allow for the rapid and specific determination of platelet, pro-coagulation, or pro-inflammatory biomarkers. Attempts to develop a reproducible test to indicate platelet activation have encountered two significant difficulties. The first is the withdrawal of blood from the patient whereby platelets become activated by the blood draw process itself. The second difficulty is that platelets then need to be separated from the withdrawn blood by centrifugation, which also can activate the platelets. Consequently, testing results may not reflect a patient's true or authentic platelet activation status. The only appropriate way to study platelet behavior in AMI patients (or possible AMI patients) is in real-time and prior to or concurrent with genuine platelet activation.

### BRIEF SUMMARY OF THE INVENTION

The present invention relates to a medical device and method for detecting acute myocardial infarction biomarkers from a blood sample.

One embodiment relates to a device for detecting biomarker analytes indicative of acute myocardial infarction or drug resistance in a fluid sample. The device includes an optical material body having a surface-textured area. A plasma polymerized layer is associated with the surface-textured area on the optical material body. An analyte-specific chemistry is coupled to the plasma polymerization layer, the analyte specific chemistry being specific for a biomarker analyte indicative of acute myocardial infarction or drug resistance. The analyte-specific chemistry has at least one optical property sensitive to binding of the biomarker analyte.

A further embodiment relates to a device for detecting biomarker analytes indicative of acute myocardial infarction or drug resistance in a fluid sample. The device includes an optical material body having a first surface-textured area and a second surface-textured area. A plasma polymerized layer is associated with the first surface-textured area and a plasma polymerized layer is associated with the second surface-textured area on the optical material body. An analyte-specific chemistry is coupled to the plasma polymerization layer associated with the first surface-textured area and an analyte-specific chemistry is coupled to the plasma polymerized layer associated with the second surface-textured area, the analyte specific chemistry for associating with a biomarker analyte indicative of acute myocardial infarction or drug resistance. The analyte-specific chemistry has at least one optical property sensitive to binding of the biomarker analyte thereto.

Another embodiment relates to a method for detecting acute myocardial infarction biomarkers or drug resistance in a patient. An optical material body having a textured surface and having elongated projections, a plasma polymerization-modified surface, and at least one analyte-specific chemistry is obtained. A fluid sample is placed on the optical material body. The fluid sample is separated into a plurality of fluid components on the optical material body, and at least one of the components contains analytes. The separated fluid component containing analytes is placed adjacent the elongated projections of the textured surface on the optical material body such that the separated component is received within the elongated projections. The separated fluid component within the elongated projections is optically sensed to detect analyte biomarkers for myocardial infarction or drug resistance.

Another embodiment includes a method for making an optical element for detecting impending myocardial infraction or drug resistance. The optical material body is etched with atomic oxygen to obtain a textured surface. A plasma polymerized layer is adhered to the textured surface by plasma polymerization. An analyte-specific chemistry is adhered to the plasma polymerized layer, the analyte-specific chemistry being specific for a biomarker analyte indicative of either acute myocardial infarction or drug resistance. The analyte-specific chemistry has at least one optical property sensitive to binding of the biomarker analyte thereto.

### BRIEF DESCRIPTION OF THE DRAWINGS

FIG. 1 is a pictorial view of a SEM image of a textured surface.

FIG. 2 is a schematic diagram of a sensor element incorporating an optical fiber having a textured surface at the tip, to which an analyte specific chemistry is attached.

FIG. 3 is a schematic diagram of the sensor element of FIG. 2 showing the separation of the blood sample.

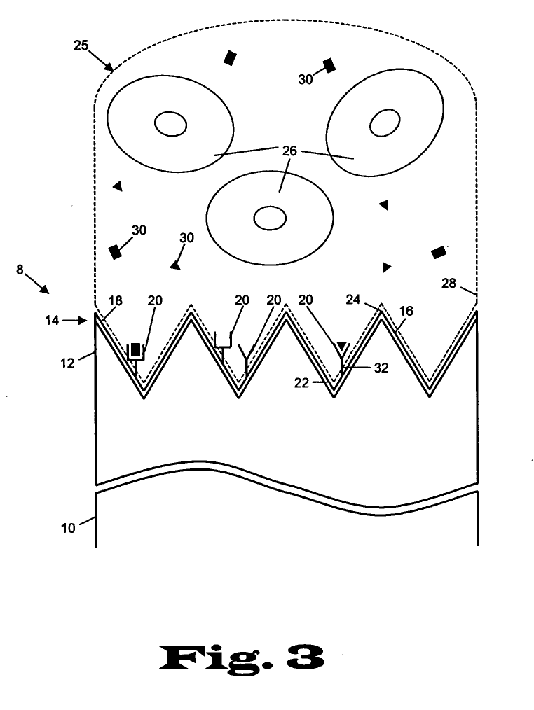

### DETAILED DESCRIPTION OF THE INVENTION

The present invention relates to devices and methods for the analysis of biological fluid samples, such as blood, for acute myocardial infarction (AMI) precursors or biomarkers, using a biosensor technology. While reference will be made to blood throughout, the fluid sample can include other biological samples, such as urine or saliva. The sensor provides for the spatial separation of the cellular elements of the blood, and provides a rapid analysis of the separated blood plasma component using reagents attached to the sensor, which are specific to the biomarker being measured. Therapeutic cardiovascular drug monitoring can also be performed with the assays. These assays can measure specific platelet and coagulation proteins that participate early in the evolution of a thrombus (blood clot) and later in a potential acute myocardial infarction (AMI). By assaying a blood sample for these AMI precursors/biomarkers, a determination can be made much earlier whether the presenting patient is a potential candidate for an AMI or whether the patient is experiencing GERD or other non life threatening symptoms.

A body fluid sample rests on the surface of an optical material of a biosensor. The surface is suitably textured so that it presents the morphology of a field of elongated projections. The projections are suitably spaced apart to exclude certain cellular components, such as blood cells, of the body fluid sample from entering into the spaces between the projections, while permitting the remaining part of the body fluid sample, which contains the analyte, to enter into those spaces. The term “analyte” is used to refer to the substance to be detected in the fluid sample.

The analyte contacts an analyte-specific chemistry on the surface of the sensor, whereupon the analyte and the analyte-sensitive chemistry interact in a manner that is optically detectable. Suitable analyte-specific chemistries include receptor molecules as well as reactive molecules. The nature and arrangement of the analyte-specific chemistry varies depending on the application. For example, the analyte-specific chemistry may be a layer of one type of chemistry or an ordered array or a finely mixed composite of different types of analyte-specific chemistries.

The biosensor may include an optical material. One type of suitable optical material is the optical fiber. The optical fiber may be a single optical fiber, or may be a bundle of optical fibers. A minimally invasive sensing device that uses a light conducting fiber having a localized textured site thereon and methods for its manufacture and use are described in U.S. Pat. No. 5,859,937, which issued Jan. 12, 1999, to Nomura, and which is incorporated herein in its entirety by reference thereto. Optical fibers may be fabricated from a variety of polymers or plastics such as polymethylmethacrylate (PMMA), polycarbonate, polysulfones, polyamide, polystyrene, polyimide, polyvinyl chloride (PVC), and from other types of optical materials such as glass, plastic, glass/glass composite and glass/plastic composite fiber waveguides. Optical fibers typically although not necessarily are provided with a cladding to support the fiber and assist in guiding light along the fiber. Prior to texturing, the fiber tip is given a desired geometric shape, which is dependent on the application and performance requirements, and which include planar surfaces either normal with respect to or otherwise angled with respect to the fiber axis, convex and concave conical surfaces, and convex and concave semi-spherical surfaces.

A textured surface may be provided on a variety of optical materials other than fibers. Another type of sensor element is made from a sheet of transparent optical material such as, for example, a polymer or plastic (including polycarbonate and polyimide), glass, and quartz glass. If sample receiving areas are desired in the sheet, they may be formed by any of various processes depending on the type of optical material. Where the material is quartz, for example, the sample areas may be etched using dry or wet etch processes. Where the material is a molded plastic, the mold may contain certain surface recesses and protrusions for forming the sample areas. The sheets may include other optical components such as lenses. Multiple sensor elements may be made from each sheet by dicing, laser cutting, stamping, or otherwise dividing the sheet. Individual sensor elements or entire sheets or parts of sheets may be incorporated into a variety of sensing instruments having a diversity of different applications.

While various surface texturing processes are available, plastic optical materials preferably are textured by etching with atomic oxygen. Generation of atomic oxygen can be accomplished by several known methods, including radiofrequency, microwave, and direct current discharges through oxygen or mixtures of oxygen with other gases. Directed beams of oxygen, such as by an electron resonance plasma beam source, may also be utilized, as set forth in U.S. Pat. No. 5,560,781, issued Oct. 1, 1996 to Banks et al., which is incorporated herein in its entirety by reference thereto. Techniques for surface texturing are described in U.S. Pat. No. 5,859,937, which issued Jan. 12, 1999, to Nomura, and which is incorporated herein in its entirety by reference thereto.

Atomic oxygen can be used to microscopically alter the surface morphology of polymeric or plastic materials in space or in ground laboratory facilities. For polymeric or plastic materials whose sole oxidation products are volatile species, directed atomic oxygen reactions produce surfaces of microscopic cones. However, isotropic atomic oxygen exposure results in polymer surfaces covered with lower aspect ratio sharp-edged craters. Isotropic atomic oxygen plasma exposure of polymers typically causes a significant decrease in water contact angle as well as altered coefficient of static friction. Atomic oxygen texturing of polymers is further disclosed and the results of atomic oxygen plasma exposure of thirty-three (33) different polymers, including typical morphology changes, effects on water contact angle, and coefficient of static friction, are presented in Banks et al., Atomic Oxygen Textured Polymers, NASA Technical Memorandum 106769, Prepared for the 1995 Spring Meeting of the Materials Research Society, San Francisco, Calif., Apr. 17-21, 1995, which hereby is incorporated herein in its entirety by reference thereto.

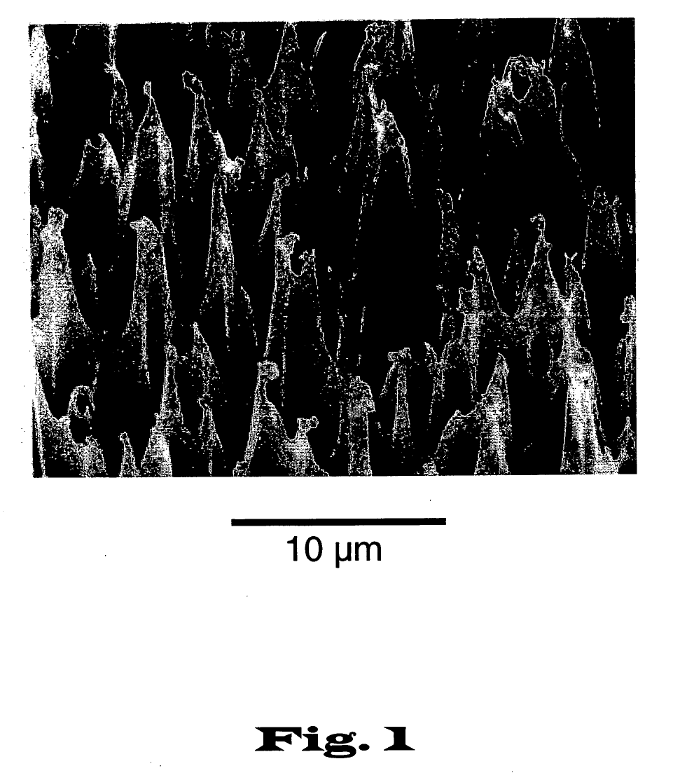

An illustrative SEM image of a textured surface as reported in the NASA Technical Memorandum is shown in FIG. 1, which shows a high aspect ratio cone-like surface morphology resulting from high fluence directed atomic oxygen exposure in space for chlorotrifluoroethylene exposed to directed atomic oxygen on the Long Duration Exposure Facility. The diameter of the cones is roughly 1 μm, the depth is roughly 5 μm, and the spacing between cones is roughly 5 μm. These dimensions are well suited for separating red blood cells from whole blood, since red blood cells tend to be of a diameter of roughly 8 μm. White blood cells are slightly larger than red blood cells.

The general shape of the projections in any particular field is dependent upon the particulars of the method used to form them and on subsequent treatments applied to them. Suitable projection shapes include, for instance, conical, ridge-like, pillared, box-like, and spike-like. While the projections may be arrayed in a uniform or ordered manner or may be randomly distributed, the distribution of the spacings between the projections preferably is fairly narrow with the average spacing being such as to exclude certain cellular components of blood, such as the red and white blood cells, from moving into the space between the projections. The projections function to separate blood components so that the analyte that reacts with the surface-resident agent on the biosensor substrate is free of certain undesirable body fluid components. In some applications, such as the ruling out of acute myocardial infarction using platelet activation markers, the spacings between the projections generally should be great enough to admit the platelets while excluding the red and white blood cells. Atomic oxygen texturing is discussed in more detail in the applications filed concurrently herewith entitled Plasma Polymerization of Atomically Modified Surfaces, and System and Apparatus for Body Fluid Analysis Using Surface Textured Optical Materials, both listing inventor Hiroshi Nomura of Shorewood, Minn., attorney docket numbers 1875.3-US-U1 and 1875.1-US-U1, respectively, which are incorporated herein by reference in their entirety. As a result of atomic oxygen texturing of the optical fiber or other optical material, ok the surface of the optical fiber/material includes a plurality of elongated projections. The optical material may include one, two, or more surface textured areas. The tip of the fiber may be textured, as well as the end of the optical fiber. The atomic surface texturing of optical materials is believed to improve sensitivity and provide an increased effective sensing area and limit background noise by supporting multiple ray reflections responsive to the light-influencing property of the analyte-specific chemistry.

The projections are suitably spaced apart to exclude certain cellular components, such as red and white blood cells, of the body fluid sample, such as blood, from entering into the wells or valleys between the projections, while permitting the remaining part of the body fluid sample, such as plasma, which contains the analyte, to enter into those wells or valleys. Analytes/biomarkers in the blood plasma, which are indicative of cellular and/or soluble platelet activation and coagulation activation, contacts or associates with the analyte specific chemistries on the surface of the elongated projections, whereupon the analyte and the analyte specific chemistry interact in a manner that is optically detectable. This permits almost instantaneous analysis of the available plasma component of blood.

The atomic oxygen textured surface on the optical fiber may be modified by plasma polymerization to allow for the adherence of the analyte specific chemistries specific for the desired analyte to be assayed. If there is more than one textured surface, one or more of the textured surfaces may be modified by plasma polymerization. Plasma polymerization and treatment are processes to modify the surface of substrate materials to achieve specific functionality. Such surfaces may be modified to become wettable, non-fouling, slippery, crosslinked, reactive, reactable and/or catalytic. The plasma polymerization process is a chemical bonding technology in which a plasma is created at or near ambient temperatures in a modest vacuum, causing a gaseous monomer to chemically modify the surface of a substrate material. Polymers obtained by the plasma process are chemically and structurally similar to starting monomers, but there are important differences. Polymerizable monomers that may be used in the practice of the invention may comprise unsaturated organic compounds such as halogenated olefins, olefinic carboxylic acids and carboxylates, olefinic nitrile compounds, olefinic amines, oxygenated olefins and olefinic hydrocarbons. Such olefins include vinylic and allylic forms. The monomer need not be olefinic, however, to be polymerizable. Cyclic compounds such as cyclohexane, cyclopentane and cyclopropane are commonly polymerizable in gas plasmas by glow discharge methods. Derivatives of these cyclic compounds, such as 1,2-diaminocyclohexane for instance, are also commonly polymerizable in gas plasmas. Particularly preferred are polymerizable monomers containing hydroxyl, amino or carboxylic acid groups. Of these, particularly advantageous results have been obtained through use of allylamine or acrylic acid. Mixtures of polymerizable monomers may be used. Additionally, polymerizable monomers may be blended with other gases not generally considered as polymerizable in themselves, such as argon, nitrogen and hydrogen. Analysis by X-ray photoelectron spectroscopy (XPS) indicates that plasma polymers form a network of highly branched and highly crosslinked segments. As an added feature, the unique mechanism of polymer formation and deposition combine to achieve excellent adhesion of the ultra-thin polymer layer to the substrate. As a result, plasma generated hydrophilic polymers are very stable in the presence of water, whereas commonly available hydrophilic polymers tend to readily dissolve in water.

In biosensor applications, affinitive materials can be prepared by plasma polymerization techniques. The development of bio-affinitive materials involves the selection of base materials, covalent coupling chemistry, and ligands. One feature of a plasma polymerization surface-modified composite sensor is its high reactivity and specific selectivity. It is standard practice to perform a blood analysis to separate plasma from whole blood via filtration techniques. This use of plasma eliminates common problems encountered when red and white blood cells are present in the sample, namely, optical interference (light absorption and light scattering) and plasma volume displacement. The resulting measurement can be significantly different from those obtained directly on whole blood.

Plasma polymerization surface-modified composite membrane sensors separate plasma from whole blood with minimal complication, and allow the direct use of whole blood as the sample for blood analysis, such that the assay may be performed with a very small amount of blood and at a much greater speed, relative to approaches that are based on membrane and wet chemistry technologies.

Although most biosensors have been designed and calibrated to be used with blood plasma, few have been built with the capability of separating plasma from a whole blood sample. The surfaces of biosensors modified by the plasma polymerization process will impart selectivity to exclude red blood cells and white blood cells and thereby promote a plasma/blood cell separation and allow the plasma to penetrate into a reactive core layer. However, current biosensors utilizing plasma modified surfaces are typically planar and the plasma polymerization process tends to remove surface irregularities and generate a smooth finished surface. Plasma polymerization is discussed in more detail in the application filed concurrently herewith entitled Plasma Polymerization of Atomically Modified Surfaces, which is incorporated herein by reference in its entirety. The textured surface at the tip of the fiber separates cellular elements (i.e., red blood cells, white blood cells) of the blood from the fluid portion (plasma) without centrifugation procedures.

FIG. 1 sets forth a biosensor element 8 incorporating an optical fiber 10 having a tip 12. Tip 12 includes an atomic oxygen textured surface 14 which includes a plurality of elongated projections 16. Tip 12 has a textured surface to which an analyte specific chemistry 20 is attached. Projections 16 may be of varying shapes or configurations. Elongated projections 16 may be treated or modified with a plasma polymer layer 18. Analyte specific chemistries 20 are associated with or coupled to, such as by attachment, chemically bound, or by physical interaction, to the plasma polymer layer 18 on the elongated projections 16, or may be attached or chemically bound to surface 14 if there is no plasma layer 18. If there are multiple surface textured areas on biosensor 8, the analyte-specific chemistry may be the same or different on each surface textured area. The analyte-specific chemistries on each surface textured area may be contiguous to one another. The analyte-specific chemistries may be of each textured surface area may be on tip 12 of optical fiber 10. As shown in FIG. 1, multiple analyte specific chemistry 20 configurations may be employed to accommodate non-symmetrical geometries of the analyte 30 stereochemistry.

The biosensor 8 can also include a polymer membrane material, such as a polyimide, instead of optical fiber 10. The membrane material can be treated with atomic oxygen texturing to generate micron morphology on the membrane. As with optical fiber 10, the membrane can be chemically modified using plasma polymerization such that the micron dimension morphology surface of the membrane is not destroyed.

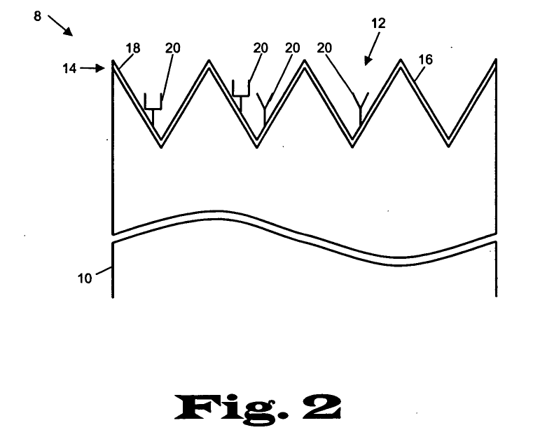

Referring to FIGS. 1 and 2, elongated projections 16 are about 3 to about 5 microns apart, and are about 5 to about 6 microns in depth from the bottom part of the well 22 to the peak 24 of the elongated projection 16. The shape and size of elongated projections 16, wells 22, and peaks 24 may vary. Spacing between elongated projections 16 allows for the separation of the cellular elements of blood or other body fluid. When a drop of blood 25 is positioned on tip 12 of sensor, the red blood cell component 26 of blood 25, as well as white blood cells (not shown), are separated from the plasma, and the red and white blood cell component is excluded from wells 22 and remains above elongated projections 16. Red blood cells are typically about 6 to about 8 microns in size, and are thus too large in fit within the spaces between the wells 22. White blood cells are slightly larger than red blood cells and also will not fit down within the spaces between the wells 22. Blood plasma component 28 settles into wells 22 between elongated projections 16 on biosensor 8. Analytes 30 contained in plasma 28 are associated with analyte specific chemistries 20, such as antibodies, enzymes, proteins, cytokines, chemokines, ligands, receptors, and peptides, thereby forming analyte-reagent complexes 32. The analytes 30 contained in the plasma 28 are then detected by optical biosensor element 8 using reflectance-based colorimetric determination of the analyte, reflectance based scattering determination of the analyte, fluorescence based determination of the analyte, chemiluminescence based determination of the analyte, or other suitable detection technique.

This optical fiber sensor system is a basic platform upon which a variety of specific assays, such as cellular and soluble platelet activation and coagulation activation assays, can take place. Analytes in the plasma can be detected very quickly with the optical methodologies, such as described above. As a result, a rapid determination can be made if new onset chest pain is a life-threatening acute coronary event or represents less threatening non-cardiac symptoms. If AMI biomarkers discussed herein are found in the fluid sample, appropriate and early interventional actions to salvage myocardial tissue at risk can be taken.

To detect acute myocardial infarction biomarkers or drug resistance in a patient, a biosensor including an optical material having a textured surface, such as an atomic oxygen textured surface, is used. The textured surface, having elongated projections, is modified by plasma polymerization. Analyte specific chemistry is coupled to the textured surface of the biosensor. A fluid sample, such as blood, is obtained, for instance by a finger stick of the patient's finger. The fluid sample is placed on the optical material body. Separation of the fluid sample into a plurality of fluid components occurs on the optical material body. One of the components contains analytes. The separated fluid component containing analytes is positioned adjacent the elongated projections of the textured surface on the optical material body such that the separated fluid component is received within the elongated projections. The separated fluid component within the elongated projections is optically sensed to detect analyte biomarkers for myocardial infarction or drug resistance.

AMI biomarker analytes that can be quickly assayed to determine whether a patient is at risk of an eventual AMI include platelet activation markers, pro-coagulation markers, pro-inflammatory markers, and cardiac markers. Platelet activation markers include, for instance, platelet membrane P-selectin (mP-selectin), Glycoprotein IIb/IIIa (GPIIb/IIIa), soluble P-selectin (sP-selectin), and soluble CD40 Ligand (sCD40L). Pro-coagulation markers include, for instance, Prothrombin fragment 1.2 (PTF1.2), D-dimer, and Thrombin Antithrombin III Binding (TAT). Pro-inflammatory markers include, for example, high sensitivity C-Reactive Protein (hsCRP) and Interleukin-6 (IL-6). Cardiac markers include Troponin I (TnI), CKMB and Myoglobin. Specialty markers include Brain Natriuretic Peptide (BNP), beta-thromboglobulin (BTG), platelet factor 4 (PF4), platelet/endothelial cell adhesion molecule 1 (PECAM-1), soluble fibrin, glycogen phosphorylase-BB, thrombus precursor protein (TPP), Interleukin-1 receptor family/ST2, Interleukin 6 (IL-6), Interleukin 18 (IL-18), placental growth factor (PIGF), pregnancy-associated plasma protein A (PAPP-A), glutathione peroxidase, plasma thioredoxin, Cystatin C, serum deoxyribonuclease I, heart type fatty acid binding protein (H-FABP), and ATP/ADP.

In addition to their role in AMI events, some of the same biomarkers may also play an active role in oncology patients. For example, the increased risk of thromboembolism in cancer patients may be related to a prothrombotic or hypercoagulable state, with abnormalities of hemostasis and platelet activation. Platelet biomarkers, indicative of platelet activation and adhesion, such as soluble and membrane-bound P-selectin and soluble CD40 ligand (sCD40L), may be increased in patients with various forms of cancer, including malignant breast cancer. This increase in platelet activation state and adhesiveness may be related to an increased risk of thromboembolism in these patients, and monitoring of these biomarkers may play a role in better management of these patients in prevention of thromboembolic events.

P-selectin, one of the above assays, indicates the onset of platelet activation and plays a vital role in the early identification of thrombus formation and subsequent acute myocardial infarction (AMI). It is one of several adhesion proteins (along with Glycoprotein IIb/IIIa) that regulate cell-to-cell attachment and is present on the surface of both activated platelets and endothelium. Activation of these cells results in the rapid expression of selectins on the cell surface that are subsequently shed into the soluble plasma pool (follow-on measurement of soluble P-selectin). Platelet P-selectin is expressed upon platelet activation due to contact with exposed collagen, thrombin or other platelet stimulants (agonists) induced by plaque rupture, leading to a potential AMI event.

Troponin I (TnI), a specific late-stage cardiac marker, is elevated in patients with infarcted myocardial tissue, and although requiring 12-24 hours post AMI before determinations are made, TnI is presently the gold standard in the diagnosis of AMI.

Platelet activation analytes, membrane P-selectin and subsequently soluble P-selectin, appear in the circulation much earlier than Troponin I (specific enzyme which leaks out of dying or dead cardiac cells) and thus provide a much more rapid and timely assessment of the potential for acute myocardial infarction to occur. These platelet activation analytes can be assayed using the device and methods described herein. The earlier an intervention, either pharmacological or mechanical, can be started to interrupt or halt the process of myocardial cell death (infarction), the greater the benefit will be to the patient. Thus, the ability to assay for early indicators of myocardial infarction is of great value.

Leukocytes, platelets, and endothelial cells interact at sites of vascular injury and inflammation through adhesion receptors on the cell surface. Since platelet adhesion to damaged or exposed blood vessels is likely to be the principal event initiating thrombus formation in vivo, assessment of altered platelet functions (e.g., glycoprotein expression, adhesiveness, aggregation) occurring as a result of ischemia provides further evidence linking platelet activation and AMI or stroke. Changes in platelet adhesiveness have been reported in patients surviving myocardial infarction. The signals (agonists) received by circulating platelets that activate platelets come from blood and the damaged blood endothelium. Agonists (stimuli) generated in blood at the site of vascular injury and capable of activating platelets include, for instance, Adenosine Diphosphate (ADP), Thrombin, Thromboxane A2, Platelet activating factor (PAF), Serotonin, Collagen and Epinephrine.

A. Cellular Response Analytes (Associated With the Cell Surface).

Cellular responses usually include platelets, monocytes and leukocytes. Platelet-leukocyte aggregates are observed in patients with unstable angina. Platelet-leukocyte heterotypic aggregates form via cell surface interactions. P-selectin on the platelet surface interacts with its receptor, PSGL-1, on the leukocyte surface and the aggregates circulate and eventually aid in stabilization of thrombi at sites of ruptured plaques. Anti-platelet therapy aims to interfere with either the formation of platelet-platelet and/or platelet-monocyte/leukocyte aggregates.

A1. Monocytes

The earliest morphologically detectable cellular event in atherogenesis is the adherence of circulating monocytes to the intact endothelial surface of large arteries. This selective recruitment of monocytes suggests that endothelium-dependent adhesion mechanisms might be responsible (Springer, TA: Nature 346:425, 1990). These leukocyte specific cell adhesion molecules (CAM's) include, for example, L-selectin, P-selectin (CD62P), E-selectin (CD62E), vascular cell adhesion molecule-1 (VCAM-1) (CD106), intercellular adhesion molecules-1 (ICAM-1), cell determinant 54 (CD54), and PECAM-1 (CD31). All of these CAM's, for instance, can be assayed using the device and methods described herein. Among these, VCAM-1 (CD106) expression on activated endothelium with its matching ligand α4 μ1 (VLA-4-CD29/49d) on activated monocytes provides a functional ligand-receptor pair that can mediate a selective adhesion event.

A2. Leukocytes

Leukocytes interact with platelets at site of arterial injury. The adhesion of leukocytes to damaged arterial surfaces is increased in the presence of platelets by a mechanism implicating platelet P-selectin. Such interactions may enhance thrombus formation and the vascular response to injury. Platelets release soluble CD40 ligand (sCD40L), a secreted activation protein, within seconds of activation in vitro and in the process of thrombus formation in vivo. CD40L on platelets induces endothelial cells to express adhesion molecules, thereby generating signals for the recruitment of leukocytes at the site of vascular injury. P-selection and P-selectin glycoprotein ligand-1 (PSGL-1) play a major role in the formation of leukocyte-platelet aggregates at the atherosclerotic site. Leukocytes bind to activated platelets through P-selectin and secure the binding with Mac-1 activation on monocytes through ICAM adhesion molecules. P-selectin and CD40L, for instance, can be assayed using the device and methods described herein.

A3. Platelets

A3.1 (GP) Ib-IX-V receptor complexes Sudden cardiac death (SCD) is one of the leading manifestations of coronary heart disease in early middle age. Platelet glycoprotein (GP) Ib-IX-V receptor complexes play a key role in the initial adhesion of platelets to collagen during the formation of a coronary thrombus. GP 1b-IX-V complex mediates platelet attachment to both endothelium and activated endothelium. It has been suggested that the HPA-2 Met/VNTR B haplotype of the platelet von Willebrand factor and thrombin receptor protein GP Ib-V-IX may be considered to be a major risk factor of coronary thrombosis, fatal myocardial infarction, and SCD in early middle age.

A3.2 Von Willebrand Factor (vWf) and Platelet Glycoproteins (GP)

Platelets are pivotal to the process of arterial thrombosis resulting in ischemia or stroke. Occlusive thrombosis is initiated by the interaction of the von Willebrand factor (vWf) and platelet glycoprotein (GP) Ib alpha. High shear stress (as in blocked arteries) facilitates vWF binding to platelet glycoprotein (GP) Ib/IX, causing activation of (GP) IIb/IIa to induce platelet aggregation. Platelet glycoproteins (GP) and von Willebrand factor (vWf) can be assayed using the device and methods described herein.

A3.3 Glycoprotein (GP) IIIa

The GPIIIa (beta3 integrin) is an integral part of two glycoprotein receptors—the GP (IIb/IIIa) fibrinogen receptors in platelets and the GP(V/IIIa) vitronectin receptors in endothelium and vascular smooth muscle cells (vSMC). The Platelet antigen (PIA) polymorphism of the gene for GPIIIa (beta3 integrin) has been suggested to play an important role in the progression of coronary artery disease (CAD) and in coronary thrombosis. The association of the PIA polymorphism with the early, non-complicated atherosclerosis and CAD was suggested in the Helsinki Sudden Death Study (HSDS). [Thromb Headmost 2000 July; 84(1): 78-82]. Glycoprotein (GP) IIIa, for instance, can be assayed using the device and methods described herein.

A3.4 Platelet Microparticles

High levels of shed membrane microparticles are detected in extracts of atherosclerotic plaques. The contents of human atherosclerotic plaques are highly thrombogenic and express high levels of tissue factor (TF). The microparticles are mostly monocytic and lymphocytic in orgin and retain about 97% of the total TF activity.

Leukocytes are a potential source of tissue factor microvesicles that adhere to platelets within a thrombus. Leukocytes and platelets are known to interact via CD15 (a leukocyte membrane-bound carbohydrates known as sialyl Lewisx SLe x) with CD62P (P-Selectin). CD15 and P-selectin interactions mediate the formation of highly procoagulant platelet aggregates containing TF particles from leukocytes. P-selectin can be assayed using the device and methods described herein.

B. Humoral Responses: Soluble Analytes.

B1. Thrombin

Thrombin is generated at the surface of damaged endothelium. Thrombin has been shown to increase many fold during cardiopulmonary bypass. When thrombin generation exceeds the body's capacity to inactivate circulating thrombin, disseminated intravascular inflammation may occur. Thrombin has a dual effect on coagulation. Its first effect is to stimulate the immediate release of TFPI (tissue factor pathway inhibitor) and tPA (tissue plasminogen activator) into the circulation, both of which have an immediatiate anticoagulant effect. The second effect is an upregulation of TF and PAI-1 (plasminogen activator inhibitor), resulting in the promotion of microvascular coagulation.

B2. Tissue Factor in Atherosclerotic Plaque

Tissue factor (TF) is the initiator of blood coagulation. Higher levels of tissue factor have been found in coronary atherosclerotic plaques of patients with unstable coronary artery disease. There is a correlation between the tissue factor content of the plaque and the increase in thrombin generation across the lesion. The higher tissue factor content found in plaques obtained from patients with unstable coronary disease was associated with a local increase in thrombin generation, thus suggesting a link with the in vivo thrombogenicity of the plaque. P-selectin can also induce tissue factor expression in monocytes. TF, for instance, can be assayed using the device and methods described herein.

B3. Thromboxane

Thromboxane biosynthesis has been shown to increase in patients with AMI receiving intravenous streptokinase. Since platelets are the major source of thromboxane A2, these findings suggest that there is marked platelet activation after coronary thrombolysis with streptokinase. The increase in platelet activation and thromboxane A2 biosynthesis may limit the therapeutic effect of intravenous streptokinase in acute myocardial infarction. Thromboxane A₂, for instance, can be assayed using the device and methods described herein.

B4. Cathepsin

In the presence of a chemoattractant, or agonist such as ADP, monocytes are capable to bind Factor X through Mac-1 (is it know what these are, or is there a general term that covers them?), which triggers azurophil granule discharge and releases cathepsin G. In a calcium-dependent reaction, cathepsin G cleaves Factor X, yielding an active protease Xa. Subsequent to Xa formation, a competent and fully functional prothrombinase is formed at the monocyte surface leading to the generation of thrombin from prothrombin. Cathepsin G (CTSG), a serine protease released from activated neutrophils, has a direct link to intravascular thrombosis and contributes to cardiovascular and cerebrovascular disease.

In addition, other analytes that can be assayed using the present invention include: CD42c (GP1 b-beta)-25 kD disulfide bonded to alpha subunit; CD42d (GPV); CD41 (GPIIb also known as alpha IIB integrin); CD61 (GPIIIa)-beta 3 subunit of GPIIb/IIIa complex (alpha 2b, beta 3); CD41/CD61 (GPIIb/IIIa complex)—receptor for fibrinogen, fibronectin, von Willebrand factor, and other adhesion proteins containing the Arg-Gly-Asp motif; CD36 (GPIV)-platelets/monocytes; CD49b (VLA-2)-platelets/monocytes-; CD51 (alpha V, beta 3)-vitronectin receptor; CD62p (P-selectin)-CD107a (LAMP-2)-lysosomal protein translocated to cell surface after activation; CD41a (GPIIb/IIIa)—intact IIb/IIIa complex; fibrinogen, von Willebrand factor, fibronectin and vitronectin receptor, Factor V11a, Factor Xa, glutathione peroxidase 1, and myeloperoxidase.

Analyte specific chemistries/marker specific protein tracers include, but are not limited to, antibodies, receptors, ligands, proteins, peptides, cytokines, chemokines, small molecules and the like. Analyte specific chemistries specific for a biomarker can be CD42c (GP1b-beta)-25 kD disulfide bonded to alpha subunit; CD42d (GPV); CD41 (GPIIb also known as alpha IIB integrin); CD61 (GPIIIa)-beta 3 subunit of GPIIb/IIIa complex (alpha 2b, beta 3); CD41/CD61 (GPIIb/IIIa complex)—receptor for fibrinogen, fibronectin, von Willebrand factor, and other adhesion proteins containing the Arg-Gly-Asp motif; CD36 (GPIV) platelets/monocytes; CD49b (VLA-2)-platelets/monocytes; CD51 (alpha V, beta 3)-vitronectin receptor; CD62p (P-selectin)-platelets; CD107a (LAMP-2)-lysosomal protein translocated to cell surface after activation, CD41a (GPIIb/IIIa)—intact IIb/IIIa complex; fibrinogen, von Willebrand factor, fibronectin and vitronectin receptor, Factor V11 a, Factor 10a, myeloperoxidase, glutathione peroxidase 1 or any other analyte markers listed herein. Analyte specific chemistries are commercially available.

In addition, platelet markers can also be used to determine aspirin resistance (sensitivity) and Plavix® resistance (sensitivity) in patients. An assay using the fiber optic biosensors herein can be used to perform therapeutic drug monitoring. Many therapeutic drugs commonly used in cardiovascular medicine such as aspirin, Plavix®, GPIIb/IIIa antagonists (ReoPro®, Integrilin®, Aggrastat®) and various heparins (native heparin and low molecular weight variants) have interactions with the platelet receptors (P-selectin and GPIIb/IIIa).

These receptors may be utilized to determine which patients should receive a particular therapeutic compound. For example, up to forty percent (40%) of the population is aspirin resistant. An assay to determine aspirin sensitivity can be performed so that patients not sensitive to aspirin 1) are not administered ineffective therapy that has bleeding side effects (GI/stomach primarily), and 2) could be started on more effective regimens such as Plavix®, an ADP (P2Y₁₂) receptor blocking anti-platelet/anti-thrombotic compound.

The receptors may also be used to monitor side effects of drugs like heparin that induces thrombocytopenia (an abnormal decrease in the number of platelets in the blood) in a substantial number of patients. In addition, these same receptors may be analyzed serially following drug administration to monitor the effectiveness of the particular therapy. Recent clinical reports have indicated that the GPIIb/IIIa antagonists ($1 billion market in aggregate) are not being dosed optimally due to the inability to accurately measure their effectiveness on platelet surface receptors.

A published abstract presented at a recent American Heart Association meeting (Zimmerman et al, AHA abstracts, 2001), demonstrated that arachidonic acid, which is converted by cyclooxygenase-1 (COX-1) in the platelet to pro-thrombotic thromboxane, will result in the expression of membrane P-selectin. The expression of membrane P-selectin is blocked by aspirin. Such an approach offers greater sensitivity to determine aspirin “resistance” than either the bleeding time or functional platelet aggregation responses.

The target for aspirin is the COX-1 enzyme that is irreversibly acetylated by aspirin for the life of the platelet (7-10 days). Thus, patients who are aspirin sensitive should not express membrane P-selectin on their platelet surface when the plasma is treated with aspirin and when the platelet is stimulated (challenged) with arachidonic acid. A baseline P-selectin stimulation with arachidonic acid can be done with plasma samples from a patient not currently taking aspirin and then adding aspirin exogenously to the plasma at 30-100 μM and repeating the test. Those patients who are aspirin resistant may still have arachidonic acid-induced P-selectin expression occurring on the platelet surface irrespective of the presence of aspirin. Additionally, patients already taking aspirin (aspirin has at least a 2 week washout period) could be tested for the lack or presence of arachidonic acid-stimulated membrane P-selectin expression as well.

This approach should also function for GPIIb/IIIa as well as P-selectin as both platelet activation epitopes (GPIIb/IIIa and P-selectin) are expressed in response to intra-platelet generation of arachidonic acid-stimulated thromboxane.

An additional and related assay would be directed at the determination of resistance to Plavix®-like drugs (P2Y₁₂ inhibitors). The P2Y₁₂ receptor is stimulated by ADP released from platelet granules or hemolysed red blood cells. ADP, like arachidonic acid, will also induce P-selectin and GPIIb/IIIa epitope expression on platelets. Epitope expression in response to ADP will be suppressed in patients sensitive to Plavix®) and presumably not suppressed in patients “resistant” to P2Y₁₂ inhibitors, such as Plavix® (Mueller, et. al. in Circulation, American Heart Association Abstracts, 2002).

It is recognized that upon the initiation of plaque rupture, platelet activation occurs locally at the site followed by coagulation reactions that occur on the phospholipid surface of platelets and serve to stabilize the platelet thrombus. Clearly, anti-platelet agents such as aspirin, ADP receptor antagonists (such as Ticlid® and Plavix®) and GPIIb/IIIa antagonists (such asReoPro®, Aggrastat®, Integrilin^{T}), have improved the outcome of patients with acute coronary syndrome (ACS). Further, anti-coagulant agents such as heparin, low molecular weight heparins and anti-thrombins have provided salutary effects in ACS patients as well. Thus, there is a tendency for physicians to treat patients with suspected ACS with anti-platelet and anti-coagulant drugs. However, these drugs carry the substantial risk of bleeding and only patients with heightened platelet activity and/or enhanced pro-coagulant activity should be treated. The assays of the present invention can be used to determine patients that are truly experiencing ACS, to provide a rule-out basis for releasing non-cardiac patients, and to provide more rapid and appropriate pharmacological therapy and monitoring of ACS patients.

Platelet and coagulation proteins have been identified and studied extensively that clearly participate in the evolution of thrombosis in ACS patients. Activated platelets express on their surface receptors known as GPIIb/IIIa (approximately 60,000 per platelet) and P-selectin (approximately 40,000 per platelet). The protein GPIIb/IIIa serves to mediate plasma fibrinogen binding and thus represents the final common pathway for platelet aggregation to occur and is the target of the GPIIb/IIIa antagonist drugs. Platelet P-selectin serves to mediate platelet/white cell aggregation which is important in not only platelet aggregate stability but also important in the initiation of pro-coagulant reactions trigged by tissue factor expression on the surface of white cells engaged (via P-selectin) with platelets. Platelet membrane P-selectin is also eventually cleaved off of the surface of activated platelets and resides in the circulation as the soluble form. Numerous clinical studies have indicated a strong correlation between the presence of soluble P-selectin in the blood and platelet activation in ACS patients.

Monitoring of GPIIb/IIIa receptor antagonists is currently accomplished by the Rapid Platelet Function Analyzer (RPFA), which was developed by Accumetrics. The RFPA determines the degree of GPIIb/IIIa receptor occupancy on a fibrinogen-coated microparticle in a whole blood sample. Penetration in the marketplace of this device has been minimal, however, even in light of the fact that the marketed GPIIb/IIIa antagonists (such as ReoPro®, Aggrastat^{E} and Integrilin®) garner in aggregate $1 billion in sales each year. Penetration of the RPFA has presumably been low due to the fact that receptor occupancy does not reflect the functional significance of GPIIb/IIIa receptor blockade.

The device of the present invention can be used for monitoring GPIIb/IIIa antagonists that is not based on receptor occupancy but instead on the functional ability of GPIIb/IIIa antagonists to suppress GPIIb/IIIa function on the surface of an activated platelet. Platelets will be activated with platelet agonists (stimulants) like ADP or collagen and expression of GPIIb/IIIa receptors determined on the surface of the platelet utilizing fiber optic assays. The functional significance of receptor expression of GPIIb/IIIa is that once expressed, these receptors now have the ability to bind plasma fibrinogen, in high concentration in the blood (4 mg/ml), and thus support platelet aggregation and eventual thrombus formation. In the presence of drugs such as ReoPro, Aggrastat and Integrilin, agonist (ADP or collagen)-induced GPIIb/IIIa receptor expression will be diminished. This assay format will be extremely useful in monitoring therapy in patients receiving GPIIb/IIIa antagonists in the cardiac catheterization laboratory, emergency room situations and in hospital wards. Furthermore, these assays will be performed serially as often infusions of such drugs are administered over a 12-24 hour time period and it is important to monitor such therapy to ensure that adequate levels of drug are being administered, and that not too much drug is present such that bleeding diatheses occur.

Heparin-induced thrombocytopenia (HIT) is a well-known complication of heparin administration but usually resolves upon discontinuation. However, a small proportion of HIT patients develop thrombosis associated with HIT, designated as HITT, which is often life-threatening and may lead to gangrene and amputations. Existing laboratory methods of confirming HIT/HITT do not distinguish between HIT and HITT. Recent reports indicate that a flow cytometric assay of the platelet activation marker CD62P(P-selectin), is useful to distinguish the effects of addition of HIT versus HITT plasma to normal blood.

In the past, heparin has been the sole anticoagulant for interventional cardiovascular procedures. Today, several alternate approaches to anticoagulate patients with documented (HIT) are under consideration, such as direct thrombin inhibitors, GPIIb/IIIa antagonists and ADP receptor blockers. Recent studies demonstrate that GPIIb/IIIa and ADP receptor inhibitors can block platelet activation induced by HIT serum/heparin, providing evidence that the mechanism of HIT may be multifactorial involving not only the generation of the heparin-PF4 or other antibodies but also involving platelet-specific processes and, potentially, the generation of proaggregatory substances. The new antiplatelet agents may be useful in the clinical management of HIT.

The assays using the fiber optic sensor, which determine P-selectin and GPIIb/IIIa receptor expression on the platelet surface, may be extremely useful in detecting emergent HIT such that therapy can be discontinued/altered and the patient's risk of overt thrombosis avoided. Literature work, as cited above, relies on cumbersome and untimely use of flow cytometric analysis, whereas a rapid point-of-care assay, such as the present invention could provide, would have profound treatment implications.

In physical form, the optical material of the sensor may be may be a single fiber, or a multiple fiber bundle, or a membrane, which may be coupled to a blood “finger-stick” lancing mechanism. The fiber optic sensor is utilized much like the dry chemistry (reagent) test strips which dominate today's diabetes self-monitoring market. However, the combined features of sampling blood, separating plasma from blood cells, and detecting color changes produced by analytes are conceptually different from anything now on the market. The fiber optic sensor has many advantages, including that a much smaller blood sample (about 0.2 microliters or less) is needed, a faster response (about 2 seconds) is obtained by eliminating the time delay caused by slow diffusion through the membrane reagent of existing strips, and the low cost of the fiber optic test strip. Also, the small sample size of whole blood may allow for frequent serial testing of patients without the need for blood transfusion. In addition, for point of care testing and for clinical laboratory applications, the one step procedure which eliminates the need to wash the sensing region prior to measurement also reduces measurement time and eliminates the wash phase in current laboratory practices.

Furthermore, the fiber optic system eliminates several problems when testing is compared to a central laboratory in the hospital. The time it takes to send blood specimens and receive test results is eliminated, and various central laboratory preparation procedures can alter the specimen and introduce errors. Timely results in real time provide better care for the patient, and testing can take place in emergency rooms, specialized sites such as oncology clinics, intensive care units, small clinics, or offices outside of metropolitan medical centers, and cardiac catheterization laboratories. It brings the testing to the patient-physician interface at the time of maximal usefulness. In critical situations, the quick specific test information can lead to prompt treatment or other diagnostic procedures.

The small size of the fiber sensor (approximately 250 p in diameter by about 2 cm in length), the small blood sample size and the disposable nature of the sensor is ideal for self-testing for blood glucose, cholesterol, lipids (LDL can be measured directly) or other components of the blood including antigens, antibodies, enzymes, tumor markers, coagulation and fibrinolytic components, infectious disease markers, red blood cells components after lysis and others. In the emergency room, a disposable sensor or an array of fiber sensors can provide important (and rapid) determinations of a number of screening tests, from routine to complex measurements, such as the platelet activation and pro-coagulation and pro-inflammatory markers as well as cardiac markers such as Troponin I.

The description of the invention and its applications as set forth herein is illustrative and is not intended to limit the scope of the invention. The invention in its broad sense is not to be considered as being limited to any particular application or to a specific sensor format, indicator composition, or surface treatment. Variations and modifications of the embodiments disclosed herein are possible, and practical alternatives to and equivalents of the various elements of the embodiments are known to those of ordinary skill in the art. These and other variations and modifications of the embodiments disclosed herein may be made without departing from the scope and spirit of the invention.

1. A device for detecting biomarker analytes indicative of acute myocardial infarction or drug resistance in a fluid sample, comprising:
a. an optical material body having a surface-textured area;
b. a plasma polymerized layer associated with the surface-textured area on the optical material body; and
c. an analyte-specific chemistry coupled to the plasma polymerization layer, the analyte-specific chemistry being specific for a biomarker analyte indicative of either acute myocardial infarction or drug resistance, and having at least one optical property sensitive to binding of the biomarker analyte thereto.

2. The device of claim 1 wherein the optical material body comprises an optical fiber.

3. The device of claim 2 wherein the surface textured area is disposed on an end of the optical fiber.

4. The device of claim 1 wherein the optical material body comprises a plurality of optical fibers.

5. The device of claim 1 wherein the optical material body comprises an optical material sheet.

6. The device of claim 1 wherein the optical material body comprises a polymer.

7. The device of claim 6 wherein the polymer material comprises polymethyl methacrylate (PMMA), polyimides, polysulfones, polyamides, polycarbonates, polystyrene, or polyvinyl chloride (PVC).

8. The device of claim 6 wherein the polymer material comprises polymethylmethacrylate (PMMA).

9. The device of claim 6 wherein the polymer material comprises polyimide.

10. The device of claim 1 wherein the surface-textured area comprises a plurality of elongated structures providing an increased effective sensing area and supporting multiple ray reflections responsive to the optical property of the analyte-sensitive chemistry.

11. The device of claim 1 wherein the surface-textured areas are atomic oxygen etched.

12. The device of claim 1 wherein the analyte-specific chemistry comprises antibodies, enzymes, proteins, cytokines, chemokines, ligands, receptors or peptides.

13. The device of claim 1 wherein the biomarker analyte comprises CD42c (GPIIb-beta)-25 kD disulfide bonded to alpha subunit; CD42d (GPV); CD41 (GPIIb; CD61 (GPIIIa)-beta 3 subunit of GPIIb/IIIa complex (alpha 2b, beta 3); CD41/CD61 (GPIIb/IIIa complex)—receptor for fibrinogen, fibronectin, von Willebrand factor, and other adhesion proteins containing the Arg-Gly-Asp motif; CD36 (GPIV) platelets/monocytes; CD49b (VLA-2)-platelets/monocytes; CD51 (alpha V, beta 3)-vitronectin receptor; CD62p (P-selectin)-platelets; CD107a (LAMP-2)-lysosomal protein translocated to cell surface after activation, CD41a (GPIIb/IIIa)—intact IIb/IIa complex; fibrinogen, von Willebrand factor, fibronectin, PECAM or vitronectin receptor.

14. The device of claim 1 wherein the biomarker analyte comprises high sensitive C-reactive protein (hsCRP), heart type fatty acid binding protein (H-FABP), myeloperoxidase (MPO), brain natriuretic peptide (BNP), P-selectin (soluble and membrane bound), soluble CD40 ligand (sCD40L), glycoprotein IIb/IIIa (GPIIb/IIIa), prothrombin fragment 1.2 (PTF1.2), D-dimer (DD), thrombin-antithrombin II (TAT), beta-thromboglobulin (BTG), platelet factor 4 (PF4), soluble fibrin, glycogen phosphorylase-BB, thrombus precursor protein (TPP), Interleukin-1 receptor family/ST2, Interleukin 6 (IL-6), Interleukin 18 (IL-18), placental growth factor (PIGF), pregnancy-associated plasma protein A (PAPP-A), glutathione peroxidase, plasma thioredoxin, Cystatin C, serum deoxyribonuclease I, ATP/ADP, troponin I (TnI), Troponin T (TnT), creatinine kinase-MB isoform (CK-MB), Factor Vila, Factor Xa, glutathione peroxidase 1 or myoglobin (MYO).

15. The device of claim 1 wherein the analyte comprises soluble CD40L.

16. The device of claim 1 wherein the analyte comprises Troponin I.

17. The device of claim 1 wherein the analyte comprises P-selectin.

18. The device of claim 1 wherein the analyte comprises GPIIb/IIIa.

19. A device for detecting biomarker analytes indicative of acute myocardial infarction or drug resistance in a fluid sample, comprising:
a. an optical material body having a first surface-textured area and a second surface-textured area;
b. a plasma polymerized layer associated with the first surface-textured area and a plasma polymerized layer associated with the second surface-textured area on the optical material body; and
c. an analyte-specific chemistry coupled to the plasma polymerization layer associated with the first surface-textured area and an analyte-specific chemistry coupled to the plasma polymerized layer associated with the second surface-textured area, the analyte specific chemistry being specific for a biomarker analyte indicative of either acute myocardial infarction or drug resistance, and having at least one optical property sensitive to binding of a biomarker analyte thereto.

20. The device of claim 19, wherein the analyte-specific chemistries of each of the surface-textured areas are identical.

21. The device of claim 19, wherein the analyte-specific chemistries of each of the surface-textured areas are different.

22. The device of claim 19, wherein the analyte-specific chemistries of each of the surface-textured areas are contiguous.

23. The device of claim 19, wherein the optical material body comprises an optical fiber.

24. The device of claim 19, wherein the optical material body comprises a plurality of optical fibers.

25. The device of claim 19, wherein the optical material body comprises an optical fiber, the optical fiber having a tip, wherein the analyte-specific chemistries of each of the surface-textured areas are on the tip of the optical fiber.

26. The device of claim 19, wherein the optical material body comprises an optical material sheet.

27. The device of claim 19, wherein the optical material body comprises a polymer.

28. The device of claim 19 wherein the analyte-specific chemistry comprises antibodies, enzymes, proteins, cytokines, chemokines, ligands, receptors or peptides.

29. The device of claim 19 wherein the biomarker analyte comprises high sensitive C-reactive protein (hsCRP), heart type fatty acid binding protein (H-FABP), myeloperoxidase (MPO), brain natriuretic peptide (BNP), P-selectin (soluble and membrane bound), soluble CD40 ligand (sCD40L), glycoprotein IIb/IIIa (GPIIb/IIIa), prothrombin fragment 1.2 (PTF1.2), D-dimer (DD), thrombin-antithrombin II (TAT), beta-thromboglobulin (BTG), platelet factor 4 (PF4), platelet/endothelial cell adhesion molecule 1 (PECAM-1), soluble fibrin, glycogen phosphorylase-BB, thrombus precursor protein (TPP), Interleukin-1 receptor family/ST2, Interleukin 6 (IL-6), Interleukin 18 (IL-18), placental growth factor (PIGF), pregnancy-associated plasma protein A (PAPP-A), glutathione peroxidase, plasma thioredoxin, Cystatin C, serum deoxyribonuclease I, ATP/ADP, troponin I (TnI), Troponin T (TnT), creatinine kinase-MB isoform (CK-MB), Factor VIIa, Factor Xa, glutathione peroxidase 1 or myoglobin (MYO).

30. The device of claim 19 wherein the analyte comprises soluble CD40L.

31. The device of claim 19 wherein the analyte comprises Troponin I.

32. A method for detecting acute myocardial infarction biomarkers or drug resistance in a patient, comprising:
providing an optical material body, the optical material body comprising a textured surface having elongated projections, a plasma polymerization-modified surface, and at least one analyte specific chemistry;
placing a fluid sample on the optical material body;
separating the fluid sample into a plurality of fluid components on the optical material body, at least one of the components containing analytes;
placing the separated fluid component containing analytes adjacent the elongated projections of the textured surface on the optical material body such that the separated component is received within the elongated projections; and
optically sensing the separated fluid component within the elongated projections to detect analyte biomarkers for myocardial infarction or drug resistance.

33. The method of claim 32 wherein the fluid is blood.

34. The method of claim 32 wherein the blood is separated into plasma and blood cellular components, the plasma containing analytes.

35. The method of claim 32 wherein the analyte-specific chemistry comprises antibodies, enzymes, proteins and other reagents.

36. The method of claim 32 wherein the analyte comprises high sensitive C-reactive protein (hsCRP), heart type fatty acid binding protein (H-FABP), myeloperoxidase (MPO), brain natriuretic peptide (BNP), P-selectin (soluble and membrane bound), soluble CD40 ligand (sCD40L), glycoprotein IIb/IIIa (GPIIb/IIIa), prothrombin fragment 1.2 (PTF1.2), D-dimer (DD), thrombin-antithrombin II (TAT), beta-thromboglobulin (BTG), platelet factor 4 (PF4), platelet/endothelial cell adhesion molecule 1 (PECAM-1), soluble fibrin, glycogen phosphorylase-BB, thrombus precursor protein (TPP), Interleukin-1 receptor family/ST2, Interleukin 6 (IL-6), Interluekin 18 (IL-18), placental growth factor (PIGF), pregnancy-associated plasma protein A (PAPP-A), glutathione peroxidase, plasma thioredoxin, Cystatin C, serum deoxyribonuclease I, ATP/ADP, troponin I (TnI), Troponin T (TnT), creatinine kinase-MB isoform (CK-MB), Factor VIIa, Factor Xa, glutathione peroxidase 1, or myoglobin (MYO).

37. A method for making an optical element for detecting impending myocardial infraction or drug resistance, comprising:
etching an optical material body with atomic oxygen to obtain a textured surface;
forming a plasma polymerized layer adherent to the textured surface by plasma polymerization; and
adhering an analyte-specific chemistry to the plasma polymerized layer, the analyte-specific chemistry being specific for a biomarker analyte indicative of either acute myocardial infarction or drug resistance, and having at least one optical property sensitive to binding of the biomarker analyte thereto.
